(19) United States
(12) Patent Application Publication
(10) Pub. No.: US 2019/0346417 A1
(43) Pub. Date: Nov. 14, 2019

(54) METHOD AND SYSTEM FOR AIR QUALITY ANALYSIS, DIAGNOSTICS, AND ENVIRONMENTAL CONTROL

(71) Applicant: Scientific Environmental Design, Inc., High Point, NC (US)

(72) Inventor: Douglas Glass Benefield, Mt. Pleasant, SC (US)

(21) Appl. No.: 16/282,109

(22) Filed: Feb. 21, 2019

Related U.S. Application Data
(60) Provisional application No. 62/671,008, filed on May 14, 2018.

(51) Int. Cl. G01N 33/00; F24F 11/76; G01N 1/22

(52) U.S. Cl. CPC G01N 33/0034; G01N 1/2273; G01N 33/0075; F24F 11/76

(57) ABSTRACT

A method and system for providing a user with real-time luminosity, temperature, indoor air quality sensing, diagnostics, analysis, and environmental control via a smart HVAC controller, a plurality of multi-sensor devices and personal health devices and gateways communicably engaged via a mesh network and the Internet. Indoor luminosity, temperature, indoor air quality and HVAC data from a residential or commercial structure as well as occupant health/wellness/activities status are collected, communicated, and aggregated in an application database via an Internet connection. An application server is operable to query the application database to provide a variety of home status, occupant status, analysis, and diagnostic reports. Reports containing detailed air quality and contaminant data could be communicated to client via email, web/mobile applications, and mobile push notifications. The application may generate one or more remediation recommendations, including configuration, settings, and automatic control of an HVAC system.

### CROSS REFERENCE TO RELATED APPLICATIONS

This application claims the benefit of U.S. Provisional Application 62/671,008, entitled “METHOD AND SYSTEM FOR INDOOR AIR QUALITY ANALYSIS AND DIAGNOSTICS,” filed May 14, 2018 and hereby incorporated by reference in its entirety.

### FIELD

The present disclosure relates to the field of indoor air quality systems; in particular, a method and system for luminosity, temperature, indoor air quality sensing, diagnostics, analysis, and environmental control.

### BACKGROUND

The level of comfort offered to a commercial or residential occupant is an important aspect of livability. The factors that influence comfort can range from the environmental temperature set by an HVAC system to the indoor air quality (IAQ). Serious threats to health include carbon monoxide, sulfur dioxide, nitrogen dioxide, ozone, particulate matter and other organic compounds. Hence, a comfort level of a residential structure or commercial building can have a significant impact on the health of the occupants. As people typically spend more than 90% of their time in indoor environments, health problems and diseases caused by poor IAQ can negatively affect livability and or Quality of Life (QoL). IAQ is an imperative variable that requires control for occupants' health, well-being, comfort, and ultimately QoL.

Conventional HVAC systems have advanced beyond just providing consistent thermal comfort. Advances in sensors, embedded devices, and networking have made it feasible to monitor and aid people in their homes and offices. Many systems have incorporated advanced technologies providing features that allow improvements in targeting energy efficiency, overall human comfort and livability. Thermostats capable of wireless communication now enable homeowners to conveniently monitor and control their comfort, anywhere and anytime. The advancement employs the Internet-of-Things (IoT) concept to give homeowners the ability to adjust the temperature of their home remotely using a mobile client, mobile phone, tablet or PC. However, the incorporation of IoT into HVAC systems to enable new capabilities, applications, and services has been limited by the heterogenous devices and products that are based on proprietary sensors, systems, platforms, networks, infrastructures, APIs, and numerous emerging and evolving connectivity protocols and standards.

IoT is global network infrastructure, linking physical and virtual objects through the exploitation of data capture and communication capabilities [EU FP7 CASAGRAS]. IoT is the connection of intelligent machines, fitted with a growing number of electronic sensors, via the Internet. IoT devices are generally constrained devices with limited computation and communication abilities. IoT is also a web-enabled data exchange that allow systems with more capacities to become smart and accessible, creating webs of objects and allowing integration of data, services and components. Smart objects, such as smart phones and smart watches, employing sensors that perform activity recognition and detect physical activities such as walking, running, climbing stairs, descending stairs, driving, cycling, can be considered IoT devices as well in the context of communication protocols.

IoT possesses a multi-layer architecture, generally divided into a perception layer, networking layer, service layer, and an application layer. A major challenge and issue associated with IoT and its evolution is interoperability. Interoperability is the ability of systems and organizations to work together. Various Standards Development Organizations (SDOs) and industrial consortiums (e.g., open connectivity forum and AIIJoyn) have been active in developing a global interoperability standard. The heterogeneity of underlying devices and communication technologies and interoperability in different layers, from communication and seamless integration of devices to interoperability of data generated by the IoT resources, is a challenge for expanding IoT solutions, generally and specially to HVAC systems for addressing occupants' health, well-being, comfort, and QoL, to a global scale, with a goal of avoiding silos and providing solutions and services that are application domain agnostic.

IoT technologies have also enabled health and wellness backend eHealth services to be delivered through mobile clients capable of displaying user-specific health/wellness information and controlling Personal Health Devices (PHDs). A growing number of connected PHDs is appearing on the market, such as health & fitness watches, blood pressure monitors, activity trackers, weighing scales and so forth. The PHDs include mobile devices themselves and or various sensors attached to the patients for sensing data, monitoring health status, and retrieving health history. The data can be health sensing data, health history, device information, and other user- or domain-specific data. Each interaction with the system among the devices requires a specific data structure to generate corresponding data. These interactions also need standardized structures to support interoperability between users and devices as well as devices and the server. The sensing devices are connected to the Internet to communicate with a server, enabling communication with a service provider, health data, management, and analysis functionalities for healthcare providers and patients. Currently, HVAC system applications or eHealth services don't include the monitoring of personal measurement data to assess or monitor the environmental effects on an occupant of a hospital, care facility, commercial or residential home, along with the customary function of controlling HVAC systems to create comfortable and healthy environments.

### SUMMARY

The following presents a simplified summary of some embodiments of the invention in order to provide a basic understanding of the invention. This summary is not an extensive overview of the invention. It is not intended to identify key/critical elements of the invention or to delineate the scope of the invention. Its sole purpose is to present some embodiments of the invention in a simplified form as a prelude to the more detailed description that is presented later.

An object of the present disclosure is a system for luminosity, temperature, indoor air quality sensing (IAQ), diagnostics, analysis, and environmental control comprising a gateway controller being operably engaged with an HVAC system, the controller having a Wi-Fi communications interface; a plurality of temperature and IAQ sensors operably engaged with the gateway controller via a mesh network, the plurality of luminosity, temperature, and IAQ sensors being operable to continuously measure one or more luminosity, temperature, and IAQ data and communicate the one or more luminosity, temperature, and IAQ sensors data to the controller; an application server operably engaged with an application database, the application server being communicably connected to the controller via the Internet connection, the controller being operable to communicate the one or more luminosity, temperature, and IAQ sensors data to the application server in real time via the Internet connection, and the application server being operable to process the one or more luminosity, temperature, and IAQ sensors data and communicate the one or more indoor luminosity, temperature, and IAQ sensors data to the database according to one or more application logic instructions; and, a client device communicably engaged with the application server, the client device being operable to run one or more instance of a luminosity, temperature, and IAQ sensors and IAQ application via a web or mobile interface, the luminosity, temperature, and IAQ sensors and IAQ application being configured to deliver IAQ and luminosity, temperature, and IAQ sensors status, analysis, and diagnostics according to the one or more IAQ data.

Another object of the present disclosure is a system for the control of a living environment comprising a personal health gateway (PHG) controller, the controller having a Wi-Fi communications interface; one or more Personal Health Devices (PHDs) operably engaged with the PHG controller via a mesh network, the PHDs being operable to continuously measure one or more health/wellness/activities data and communicate the one or more data to the PHG controller; an application server operably engaged with an application database, the application server being communicably connected to the PHG controller via the Internet connection, the controller being operable to communicate the one or more PHDs data to the application server in real time via the Internet connection, and the application server being operable to process the one or more PHD data and communicate the one or more PHD data to the database according to one or more application logic instructions; and, a client device communicably engaged with the application server, the client device being operable to run one or more instance of a PHD application via a web or mobile interface, the PHD application being configured to deliver one or more health/wellness/activities status of one or more occupants according to the one or more PHD data.

Another object of the present disclosure is a system for indoor luminosity, temperature, and IAQ sensors, air quality sensing, diagnostics, analysis, and environmental control comprising one or more gateway, PHG, or combinations thereof controllers being operably engaged with an HVAC system, the controllers operably engaged to continuously measure one or more luminosity, temperature, and IAQ sensors data inputs or one or more PHD inputs and communicate the one or more IAQ data inputs or PHD inputs to an HVAC system controller to create a comfortable environment with an acceptable IAQ by regulating indoor air parameters, including but not limited to air temperature, relative humidity, air speed, and chemical species concentrations in the air; an application server operably engaged with an application database, the application server being communicably connected to the HVAC controller via the Internet connection, the controller being operable to communicate the one or more said HVAC controller gateway or PHGs or combinations thereof to the application server in real time via the Internet connection, and the application server being operable to process one or more occupant's health/wellness/activities/wellness data, one or more indoor air parameters, including but not limited to air temperature, relative humidity, air speed, and chemical species concentrations in the air, and communicate the data to the database according to one or more application logic instructions; and, a client device communicably engaged with the application server, the client device being operable to run an instance of an indoor environmental condition application via a web or mobile interface, the indoor environmental condition application being configured to deliver indoor environmental condition, analysis, and diagnostics according to the one or more said HVAC controller gateway or PHG data.

Specific embodiments of the present disclosure provide for an indoor air quality control system comprising a controller being operably engaged with an HVAC system, the controller having a wireless communications interface; a plurality of indoor air quality sensors operably engaged with the controller via a mesh network, the plurality of indoor air quality sensors being operable to continuously measure one or more indoor air quality data inputs and communicate the one or more indoor air quality data inputs to the controller; an application server operably engaged with an application database, the application server being communicably connected to the controller via the Internet connection, the controller being operable to communicate the one or more indoor air quality data inputs to the application server in real-time via the Internet connection, and the application server being operable to process the one or more indoor air quality data inputs and communicate the one or more indoor air quality data inputs to the database according to one or more application logic instructions; and, a client device communicably engaged with the application server, the client device being operable to run an instance of an indoor air quality application via a web or mobile interface, the indoor air quality application being configured to deliver indoor air quality status, analysis, and diagnostics to the web or mobile interface via the indoor air quality application according to the one or more indoor air quality data inputs.

Further specific embodiments of the present disclosure provide for an indoor air quality control system comprising a controller being operably engaged with an HVAC system, the controller having a wireless communications interface; a plurality of indoor air quality sensors operably engaged with the controller via a mesh network, the plurality of indoor air quality sensors being operable to continuously measure one or more indoor air quality data inputs and communicate the one or more indoor air quality data inputs to the controller; a transceiver hub communicably engaged with the controller and the plurality of indoor air quality sensors; a system control interface operably engaged with the transceiver hub and the controller; an application server operably engaged with an application database, the application server being communicably engaged with the transceiver hub via a wireless communications interface, the transceiver hub being configured to communicate the one or more indoor air quality data inputs to the application server in real-time via the wireless communications interface, and the application server being configured to process the one or more indoor air quality data inputs and communicate the one or more indoor air quality data inputs to the database according to one or more application logic instructions; and, a computing device communicably engaged with the transceiver hub and the application server, the client device being operable to run an instance of an indoor air quality application via a web or mobile interface, the indoor air quality application being configured to deliver indoor air quality status, analysis, and diagnostics to the web or mobile interface via the indoor air quality application according to the one or more indoor air quality data inputs.

Further specific embodiments of the present disclosure provide for an occupant-centric environmental control system comprising a controller being operably engaged with an HVAC system, the controller having a wireless communications interface; a plurality of indoor air quality sensors operably engaged with the controller, the plurality of indoor air quality sensors being operable to continuously measure one or more indoor air quality data inputs and communicate the one or more indoor air quality data inputs to the controller; a personal health device being configured to measure personal health or wellness data from a user; a personal health gateway device operably engaged with the personal health device, the personal health device being configured to communicate the personal health or wellness data to the personal health gateway device, the personal health gateway device being configured to communicate the personal health or wellness data to the controller; and, an application server operably engaged with an application database, the application server being communicably engaged with the personal health gateway device and the controller via a wireless communications interface to receive the personal health or wellness data and the indoor air quality data and store the personal health or wellness data and the indoor air quality data in the application database, the application server being configured to process the health and wellness data and the indoor air quality data to define one or more environmental correlations between the health and wellness data and the indoor air quality data, and application server being configured to define one or more HVAC system controls according to the one or more environmental correlations.

The foregoing has outlined rather broadly the more pertinent and important features of the present invention so that the detailed description of the invention that follows may be better understood and so that the present contribution to the art can be more fully appreciated. Additional features of the invention will be described hereinafter which form the subject of the claims of the invention. It should be appreciated by those skilled in the art that the conception and the disclosed specific methods and structures may be readily utilized as a basis for modifying or designing other structures for carrying out the same purposes of the present invention. It should be realized by those skilled in the art that such equivalent structures do not depart from the spirit and scope of the invention as set forth in the appended claims.

### BRIEF DESCRIPTION OF DRAWINGS

The above and other objects, features and advantages of the present disclosure will be more apparent from the following detailed description taken in conjunction with the accompanying drawings, in which:

FIG. 1A is a system architecture diagram of an indoor air quality analysis and diagnostics system, according to an embodiment of the present disclosure;

FIG. 1B is a system diagram of a luminosity, temperature, indoor air quality analysis and diagnostics system, according to an embodiment of the present disclosure;

FIG. 2A is a functional block diagram of a routine for indoor air quality status, according to an embodiment of the present disclosure;

FIG. 2B is a data processing flow for indoor air quality status, according to an embodiment of the present disclosure;

FIG. 3 is a functional block diagram of a routine for indoor air quality analysis, according to an embodiment of the present disclosure;

FIG. 4 is a functional block diagram of a routine for indoor air quality analysis, according to an embodiment of the present disclosure;

FIG. 5A is a functional block diagram of a routine for indoor air quality diagnostics, according to an embodiment of the present disclosure;

FIG. 5B is web application screenshots of an indoor air quality diagnostics, according to an embodiment of the present disclosure;

FIG. 6 is a functional block diagram of a routine for indoor air quality remediation, according to an embodiment of the present disclosure;

FIG. 7 is a system architecture diagram of an indoor air quality analysis, diagnostics, and HVAC control system incorporating a personal health device, according to an embodiment of the present disclosure;

FIG. 8 is a data processing flow for occupant health/wellness/activities status, according to an embodiment of the present disclosure;

FIG. 9 is a functional block diagram of a routine for occupant health/wellness/activities data analysis, according to an embodiment of the present disclosure; and,

FIG. 10 is a functional block diagram of a routine for occupant environment analysis, according to an embodiment of the present disclosure.

### DETAILED DESCRIPTION

Exemplary embodiments are described herein to provide a detailed description of the present disclosure. Variations of these embodiments will be apparent to those of skill in the art. Moreover, certain terminology is used in the following description for convenience only and is not limiting. For example, the words “right,” “left,” “top,” “bottom,” “upper,” “lower,” “inner” and “outer” designate directions in the drawings to which reference is made. The word “a” is defined to mean “at least one.” The terminology includes the words above specifically mentioned, derivatives thereof, and words of similar import.

Embodiments of the present disclosure enable a method and system for providing a user with real-time luminosity, temperature, and IAQ sensors, IAQ sensing, diagnostics, analysis, and environmental control via a smart HVAC controller, a plurality of luminosity, temperature, and IAQ sensors devices, one or more PHDs, communicably engaged with each other and the smart HVAC controller via a mesh network, one or more gateways, and the Internet. Luminosity, temperature, and IAQ sensors, and occupant health/wellness/activities data and HVAC data from a residential or commercial structure are collected, communicated, and aggregated in an application database via an Internet connection. An application server is operable to query the application database to provide a variety of home or occupant health/wellness/activities status, analysis, and diagnostic reports. Luminosity, temperature, and IAQ sensors, indoor air quality or occupant health/wellness/activities data reports are delivered to a user via a web or mobile-based reporting application. In the event of occupant environmental concerns, the user could add IAQ monitoring sensors as needed to isolate and diagnose the cause and location of IAQ issues. Reports containing detailed luminosity, temperature, and IAQ sensors, air quality and contaminant data could be communicated to the client via email, web/mobile applications, and/or mobile push notifications. The application may be operable to generate one or more remediation recommendations, including configuration of HVAC controls and system settings and remote automated control of HVAC operations.

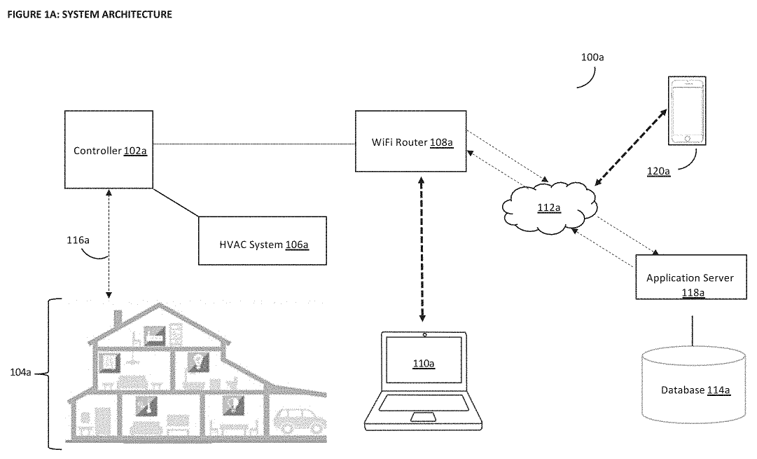

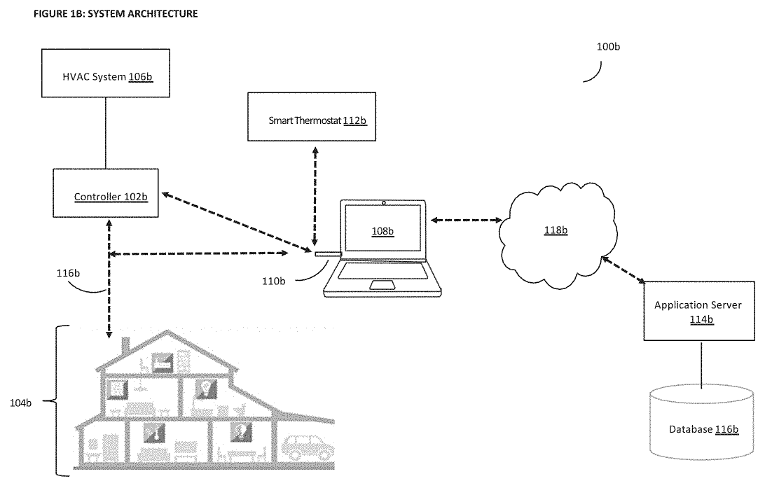

Referring now to FIG. 1a, a system diagram of a luminosity, temperature and an IAQ analysis and diagnostics system 100a is shown. According to an embodiment of the present disclosure, luminosity, temperature, and IAQ analysis and diagnostics system 100a is generally comprised of a smart HVAC controller 102a, a plurality of multi-sensor luminosity, temperature, and IAQ devices 104a, an HVAC system 106a operably engaged with controller 102a, a Wi-Fi router 108a, and an application server 118a operably engaged with an application database 114a. Controller 102a is installed in the interior of a commercial or residential building and may be configured to control one or more functions of HVAC system 106a. Controller 102a may be configured as a Wi-Fi enabled thermostat, including one or more integrated luminosity, temperature and IAQ sensors. Controller 102a is coupled to application server 118a via Wi-Fi router 108a over Internet connection 112a. Multi-sensor IAQ devices 104a may be distributed in one or more rooms of the commercial or residential building; for example, if a building has six rooms, there may be a multi-sensor IAQ device 104a installed in each room. Multi-sensor IAQ devices 104a may be distributed between living spaces and mechanical spaces in the commercial or residential building: for example, inside the vents including air returns, integrated within the thermostat, portable battery powered units, wall outlet plug-ins, and the like. Multi-sensor IAQ devices 104a may also be placed or installed outside the commercial or residential building to correlate outdoor air quality against IAQ. Outside air can be a source of contaminants such as mold or pollen, or can be a source of clean fresh air if, for example, the IAQ is worse than the outdoor air quality. This would enable HVAC systems that incorporate a fresh air intake to selectively engage and disengage such intake in response to IAQ data.

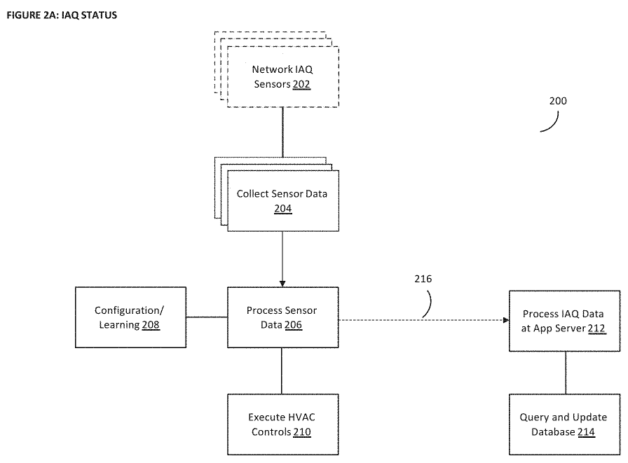

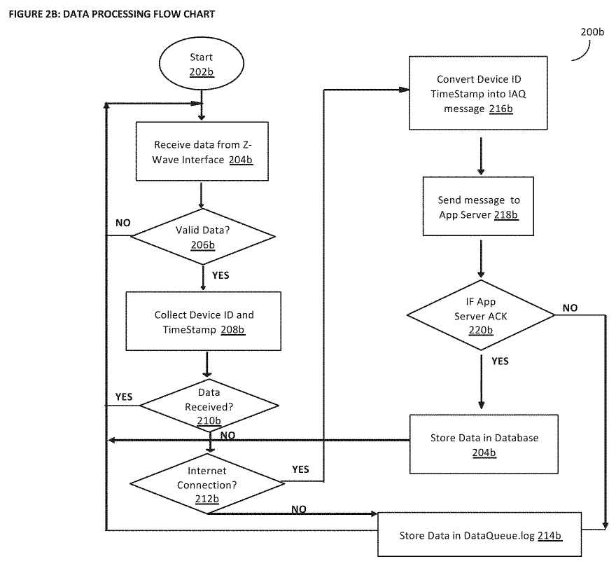

Each multi-sensor IAQ device 104a may be operably engaged with each other to communicate data to controller via mesh network 116a. Multi-sensor IAQ devices 104a may be comprised of one or more sensors being operable to measure a plurality of IAQ data points, such as luminosity (LUX), temperature, humidity, PM2.5 (particulate matter), carbon dioxide, volatile organic compounds, sound (audible or ultrasonic), light intensity, smoke, or the like. Each multi-sensor IAQ device 104a may be assigned a unique identifier (e.g., IP address, etc.) corresponding to a location in the building, such that data collected by multi-sensor IAQ devices 104a may be associated with a specific location in the building or residential structure. Multi-sensor IAQ devices 104a are configured to continuously collect data and communicate such data to controller 102a via mesh network 116a in real-time. Controller 102a communicates data to application server 118a via Internet connection 112a. Application server 118a processes data via application logic, and routes IAQ data to database 114a. Database 114a aggregates and stores IAQ data and associates it with a date/time stamp and the device identifier. Application server 118a is operable to query database 114a to assemble one or more IAQ reports comprising status, analysis, and diagnostics. The one or more IAQ reports are assembled in a user interface and accessed on a client device 110a via a web browser or mobile interface on a mobile device 120a. Embodiments of system 100a are operable to execute routines 200 through 600, as described in FIGS. 2-6 below.

Referring now to FIG. 1b, a system diagram of a luminosity, temperature, and IAQsensors and an IAQ analysis and diagnostics system 100b is shown. According to an embodiment of the present disclosure, luminosity, temperature, and IAQ sensors and IAQ analysis and diagnostics system 100b is generally comprised of a smart HVAC controller 102b, a plurality of multi-sensor luminosity, temperature, and IAQ sensors and IAQ devices 104b, an HVAC system 106b operably engaged with controller 102b, computing unit 108b connected to a USB transceiver hub 110b, a thermostat 112b and an application server 114b operably engaged with an application database 116b. USB transceiver hub 110b contains a wireless communication protocol residing in firmware for controlling mesh network communication with the plurality of multi-sensor luminosity, temperature, and IAQ sensors and IAQ devices 104b and thermostat 112b. Controller 102b is installed in the interior of a commercial or residential building and may be configured to control one or more functions HVAC system 106b. Controller 102b is coupled to application server 114b via computing unit 108b over Internet connection 118b through one or more communication channels including but not limited to wireless (e.g., Wi-Fi), Ethernet, or the like. In various embodiments, USB transceiver hub 110b comprises, but is not limited to, a Z-Wave USB (Aeotec Stick GenS-Aeotec, El Cerrito, Calif.). In various embodiments, IAQ devices 104b comprise, but are not limited to, a Z-Wave compatible A8-7 Multisensor monitor (Guangzhou MCO Home Technology Co., Ltd.), capable of sensing Temperature: in the range of about −20° C. to about 100° C., Humidity: in the range of about 0% RH to about 99% RH, PM2.5: in the range of about 0 ug/m3 to about 500 ug/m3, CO2: in the range of about 0 ppm to about 2000 ppm, Illumination: in the range of 0 Lux to about 40000Lux, Noise: in the range of about 30 dB to about 100 dB, and HCHO: in the range of about 0 ug/m3 to about 1000 ug/m3. In various embodiments, IAQ devices 104b comprise a Z-Wave compatible MH10 Monitor (Guangzhou MCO Home Technology Co., Ltd.) capable of sensing PM2.5 value in air with high accuracy (PM2.5 detection range: about 0 ug/m3 to about 999.9 ug/m3), Temperature range: −9.0° C. to about 50° C., and Humidity range: 0% RH to about 99% RH. In various embodiments, thermostat 112b comprises, but is not limited to, a Z-Wave compatible RCS TZ45 Thermostat (RCS, Poway, Calif.). In various embodiments, computing unit 108b communicates and controls, via USB transceiver hub 110b, one or more said multisensory monitors through mesh network 116b. One or more multi-sensor IAQ devices 104b may be paired with computing unit 108b and USB transceiver hub 110b through an inclusion process. In an alternative embodiment, computing unit 108b may add all discovered devices within physical proximity without discrimination. In an alternative implementation, the hardware, firmware, and software of one or more controller 102b, computing unit 108b, USB transceiver hub 110b, may be combined into a single unit to control the function of HVAC system 106b.

The gateway controller/router computing unit 108b, smart hub 110b, thermostat 112b, and IAQ devices 104b may comprise alternative IoTs, hardware, firmware, software, physical network, network protocols, data protocols, architecture, framework, standards, applications, and APIs to sense, diagnose, analyze, and transmit data to application server 114b and database 116b. These alternative implementations include but are not limited to TSN Ethernet (802.1, 802.3) Wireless PAN (802.15, 802.15.4), Wireless LAN (802.11 Wi-Fi), Wireless 2G/3G/4G/5G/LTE, Wireless WLAN (802.16), Thread, Zigbee, WirelessHART, Bluetooth, BLE, Wi-Fi, LTE, Internet Protocol (IP), IPv6, IPv6 over Low-Power Wireless Area Network (6LoWPAN), IP6 over BLE, TCP, UDP, TCP, DDSI-RTPS, CoAP, MQTT, NFC, HTTP, DDS, TLS, DTLS, oneM2M, Web Services, GATT protocol, ATT protocol, Representational State Transfer (REST) APIs, Lightweight M2M, SOAP, HL7, HL7 CDA, IEEE 11073 DIM, or the like. Exemplary platforms include but are not limited to TI CC2538, nFR52832, TI MSP430x, Atmel AVR, Freescale MC1322x, Arduino, Quark D200, CC2650, NXP FRDM, Hexiware, nRF52, ST Nucleo, Imote2, Shimmer, IRIS, Telos Rev B, MicaZ, Mica2, Mica2dot, Mulle, TinyNode, Zolertia Z1, UCMote Mini, nRF52840, nRF51 DK, BMD-300-EVAL-ES, STM32F4DISCOVERY, STM32-E407, Arduino Zero, Arduino Zero Pro, NUCLEO-F401RE, PIC32MX470, PIC32MZ2048EFG100, Arduino-due, UDOO, CC2538DK, OpenMote, pca10005, yunjia-nrf51822, STM32 Nucleo32, telosb, chronos, Altera, Atmel, Cortus, Freescale, Infineon, Microsemi, NXP, Renesas, TI, ST, Intel, Xilinx, Nordic nRF52DK, Seed Arch Link, Realtek RT8195AM, Wizwiki, EFM32, NUCLEO F334R8, hexiwear, mbuino, mbedLPC.

Referring now to FIG. 2, a functional block diagram of a routine 200 for IAQ status is shown. According to an embodiment of the present disclosure, routine 200 for IAQ status is initiated to measure and communicate to a user an IAQ status for a commercial or residential building. IAQ sensors 202 are installed in one or more rooms of the commercial or residential building. In a preferred embodiment, IAQ sensors are installed in at least every room of the commercial or residential building. IAQ sensors are operably engaged via a mesh network, and sensor data is collected and communicated to the controller 204. The sensor data is processed at the controller level 206 and is communicated to the application server via communications interface 216. The application server processes the IAQ data 212 and queries and updates the database 214 with the data. The controller may also execute system configurations and/or machine learning steps 208 in response to the sensor data. System configurations and/or machine learning steps may be utilized to execute one or more HVAC controls 210 via the controller. For example, if IAQ is reduced at certain times of day, the controller may execute instructions for the HVAC system to run the fan or increase the fresh air intake to proactively maintain desired IAQ thresholds in response to data trends.

Referring now to FIG. 2b, a flow chart of data processing by a gateway controller of the HVAC system is shown. At the Start 202b, controller 204 receives data 204b from at least one IAQ device 104b via Z-wave interface of USB transceiver hub 110b of FIG. 1b. The validity of sensors data is determined at step 206b, a NO decision leading to the initiation of another query from Start 202b. If a YES determination is made at 206b, then smart hub 110b proceeds to collect a Device ID and TimeStamp 208b from an IQA device 104b. Another decision is made at 210b whether the collected data has been received before. If NO, then smart hub 110b proceeds to determine whether Internet Connection 212b is available. If NO, then smart hub 110b proceeds to the Store Data 214b step where data is stored in a queue file for later transmission to application server 118a. If YES, then smart hub 110b proceeds to convert the data into an IAQ message at step 216b and then sends (Step 218b) the message to application server 118a. A decision is made at 220b to confirm that application server has received data. If a NO acknowledgement is received, then smart hub 110b stores the data at 214b for later transmission. If smart hub 110b receives a YES acknowledgement, then the hub proceeds to query another IAQ device 104b for data. In various embodiments, instructions or steps for sensing, measurement, or IAQ data processing mode can be stored or updated in firmware within one or more smart hub 110b, thermostat 112b, and IAQ devices 104b. In various embodiments, firmware instructions can include but are not limited to calling software libraries, initializing sensors, initializing device power, reading device IDs, pairing devices, determining sensor warmup time, acquiring sensor outputs, calibrating sensors, fault detection, collecting timestamps, sending data, and processing one or more recursive instructions.

Referring now to various embodiments of the application layer, including but not limited to applications stored in application server 114b, data is posted from smart hub 110b using one or more APIs. In various embodiments, one or more scripts manages real-time acquisition, processing, and analysis of messages for subsequent storage to database 114a. In an embodiment, a posting script, posting API returns a response to the result of the post. The process checks whether data has been received successfully from one or more IAQ devices of 104b, stored on a computing stack. One or more scripts running, for example, on computing unit 108b, can check the status of one or more stack, and manage a mechanism of message transmission or re-submission depending on the status of Internet connection. In various embodiments, the message transfer protocol for one or more IAQ devices 104b includes but is not limited to RESTful, compatible with HTTP for devices with limited resources such as battery capacity, low memory, or reduced processing capabilities. In various embodiments, the application layer protocol includes the constrain application protocol (CoAP). In various embodiments, IAQ devices 104b, controller 102a, smart hub 110b, or thermostat 112b may communicate via messages with application server 118a using protocol layer such as Message Queue Telemetry Transport (MQTT) to connect devices with middleware and real-time applications. In various embodiments, connect devices achieve real-time functions through the binding, bridge (e.g., ponte), or broker (e.g., MOTT broker) of one or more said protocols (e.g., CoAP, HTTP, MQTT, DTLS, UDP, XMPP, SMS, Web Socket, etc.). In various embodiments, Device Management (DM) of IAQ devices 104b, controller 102a, smart hub 110b, or thermostat 112b comprises the use of OAM DM or OMA Lightweight M2M (LWM2M) protocols or standards. DM functionalities include, but are not limited to; bootstrapping to automatically connect one or more IAQ sensing devices 104b to application server 118a using key management, device configuration to change parameters of the device and network settings, firmware updates, fault management for automatic error reporting, debugging, configuration, control applications, reporting, notification mechanism to alert for new sensor values, alarms and events. In various embodiments, IAQ devices 104b, controller 102a, smart hub 110b, thermostat 112b, or application server 118a employ the ETSI M2M standard interfaces (mla, dla and mid) and Service Capabilities Layers at one or more device, gateway, or network domains to achieve IoT interoperability. In various embodiments, one or more interworking proxy enables devices non-compliant with ETSI M2M by translating one or more specific protocol to another protocol (e.g., CoAP message to a specific HTTP POST message, etc.). In a various embodiment, real-time operation employs ETS M2M standard (e.g., NSCL, GSCL) whereby one or more IAQ devices 104b reports data to the GSCL enabling high-level applications at the network domain to retrieve data via NSCL. In various embodiments, application server 118a interacts with the GSCL via NSCL to discover registered applications (e.g., IAQ sensing device or node) to create a subscription to the particular resource. Application server 118a monitors one or more IP or port for incoming data. In one embodiment, IAQ devices 104b, controller 102a, smart hub 110b, or thermostat 112b, transmit new data, the GSCL automatically sending an HTTP POST with a, but not limited to, XML encoded message containing a new reported data value to server 118a.

An object of the present disclosure is a real-time monitoring of web applications via application server 118a. In various embodiments, one or more software module, including but not limited to Node.js modules, are adapted to process a plurality of data corresponding to luminosity, temperature, indoor air quality sensing, diagnostics, analysis, and environmental control. In various embodiments, one or more Node.js modules employ an event loop, a thread pool, or combinations thereof. In one embodiment, the event loop is a single thread application. In another embodiment, input-output (I/O) functions are delegated to a thread pool under the control of an operating system (OS). In a preferred embodiment, the event loop continuously retrieves code from an event queue and executes it, excluding callbacks from previously stacked I/O tasks. Upon the completion of a previous I/O task, the event loop processes any callback. In various embodiments, the said software modules are fast and scalable I/O bound applications containing simple low-level complex event loop and OS callbacks abstractions for built-in real time web monitoring. In various embodiments, processing time is reduced using one or more interworking proxy, including but not limited to, MQTT-binding and Web Sockets, ponte to bridge CoAP to MQTT, one or more MQTT broker, to bridge HTTP, CoAP, and MQTT messages, services, or the like.

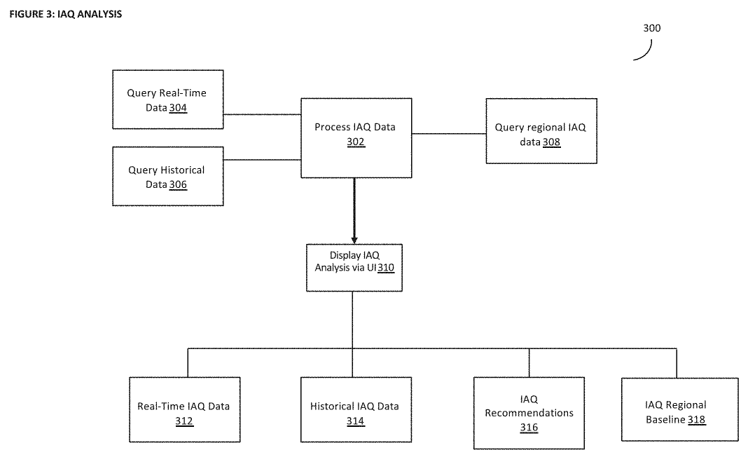

Referring now to FIG. 3, a functional block diagram of a routine 300 for IAQ analysis is shown. According to an embodiment of the present disclosure, routine 300 for IAQ analysis is initiated to process IAQ data at the application server level 302. The application server executes instructions to query real-time IAQ data from the application database 304, and query historical IAQ data from the application database 306. The application server may also query regional or geographically proximal third-party IAQ data 308 to aggregate such data and provide a baseline average of various air quality measurements (e.g. PM2.5 (particulate matter), carbon dioxide, volatile organic compounds, smoke, and the like). The application server processes the queried data 302 to assemble an IAQ analysis for a specific residential or commercial building and communicates and displays the analysis via a user interface 310 executing on a client device. The IAQ analysis provides one or more recommendations and data visualizations for the user, including real-time IAQ data 312, historical IAQ data for the subject building 314, IAQ data recommendations based on real-time and historical data 316, and IAQ comparisons between the subject building and regional baseline data 318.

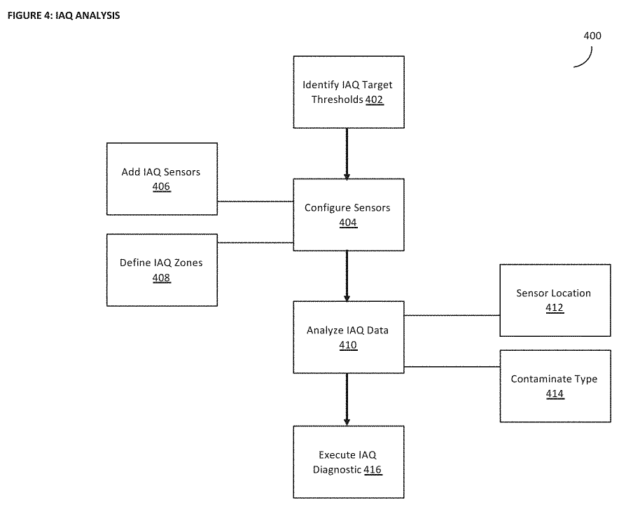

Referring now to FIG. 4, a functional block diagram of a routine 400 for IAQ analysis is shown. According to an embodiment of the present disclosure, routine 400 is initiated to execute an IAQ analysis in response to the IAQ analysis of routine 300, as discussed above. Routine 400 comprises identifying target thresholds for one or more IAQ measurements (e.g. PM2.5 (particulate matter), carbon dioxide, volatile organic compounds, smoke, and the like). Sensors are configured in one or more rooms of the residential or commercial building 404 to measure IAQ data. If recommended pursuant to routine 300 above, additional sensors can be added to the residential or commercial dwelling 406, and additional IAQ zones can be defined 408 and associated with the sensors to provide location-specific IAQ monitoring. The system analyzes the IAQ data 410 and aggregates the data according to sensor location 412 and contaminate type 414 (e.g. PM2.5 (particulate matter), carbon dioxide, volatile organic compounds, smoke, etc.). The IAQ data is processed and an IAQ diagnostic 416 is then executed by the application server in communication with the application database.

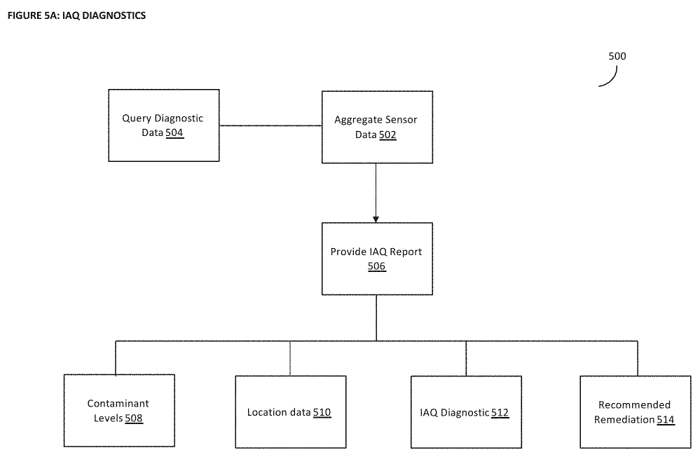

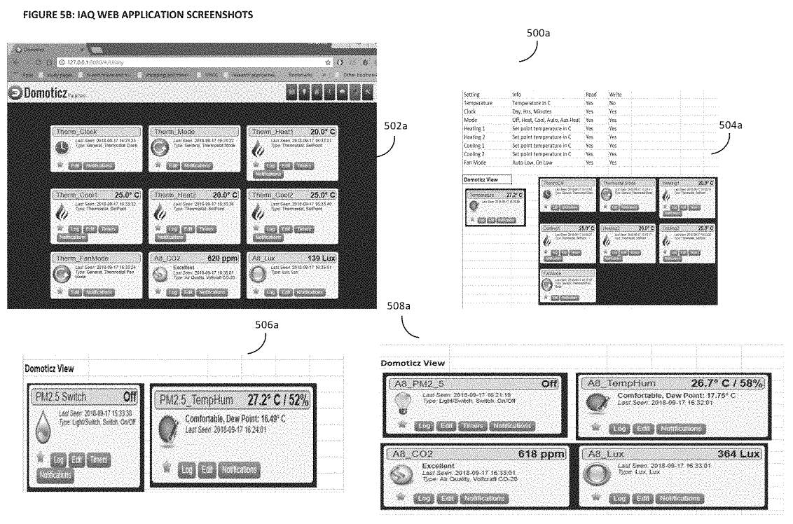

Referring now to FIG. 5, a functional block diagram of a routine 500 for luminosity, temperature, and IAQ sensors and IAQ diagnostics is shown. According to an embodiment of the present disclosure, routine 500 is initiated to execute luminosity, temperature, and IAQ sensors and an IAQ diagnostic in response to the luminosity, temperature, and IAQ sensors and IAQ analysis of routine 400, discussed above. The application server queries diagnostic data 504 and aggregates sensor data 502. The application server executes application logic to assemble an IAQ report 506 pursuant to the sensor data and diagnostic data. The luminosity, temperature, and IAQ sensors and IAQ report is delivered to the user via the web or mobile application to provide the user with targeted insights and data visualizations with respect to contaminant levels 508, contaminant location data 510 (i.e., source of contamination), a detailed IAQdiagnostic 512 (i.e., problem and cause), and one or more recommended remediation steps 514 (i.e. solution to mitigate IAQ problems). The recommended remediation 514 may be a mix of automated steps (i.e., system configuration and controls) and manual/physical steps (i.e., new HVAC, increased fresh air intake, etc.). In various embodiments, the client-side data visualization is built using one or more JavaScript frameworks, including but not limited to Angular, Express, D3.js, or the like.

Referring now to FIG. 5a, exemplary web application output screen captures 500a generated from using the computing unit 108b described in FIG. 1b are shown. An open source application Domiticz (https://www.domoticz.com) enables the monitoring and configuration of various Z-Wave compatible luminosity, temperature, and IAQ sensor devices 104b and thermostat 112b by downloading the software for operation in conjunction with computing unit 108b. The open source application user-interface is a scalable HTML5 web frontend and is automatically adapted for Desktop and Mobile Devices. Notifications and alerts can be sent to any mobile device, for example, mobile client device 120a of FIG. 1a. Screen capture 502a provided evidence that all IAQ sensors including luminosity, fan mode, and thermostat data were detected by the Z-Wave hub 110b and all data accessible. In addition, all data from Z-Wave thermostat 112b was accessible and readable, demonstrated with screen shot 504a, and transmitted and recorded in database 114a of FIG. 1a. In one example, screen shot 506a shows that data from Z-Wave compatible MH10 Monitor is readable and detectable; with PM2.5 selectively switched off. In another example, screen shot 508a show that data from Z-Wave compatible A8-7 Multisensor monitor is readable and detectable; with PM2.5 selectively switched off.

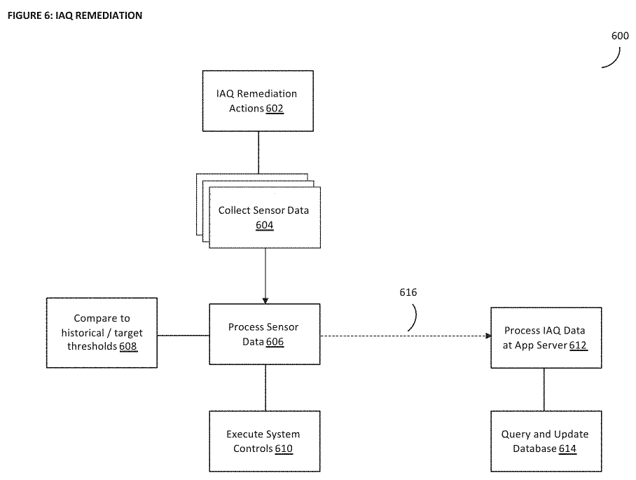

Referring now to FIG. 6, a functional block diagram of a routine 600 for IAQ remediation is shown. According to an embodiment of the present disclosure, routine 600 for IAQ remediation is initiated to provide an IAQ status in response to one or more executed remediation actions. The system and/or the user may execute steps to take one or more remediation actions as provided in routine 500, above (e.g., automated steps such as system configuration and controls, modulation of air temperature, relative humidity, or air speed, and/or manual/physical steps such as new HVAC, increased fresh air intake, and the like). The system collects sensor data 604 via the IAQ monitoring devices and processes the sensor data 606 at the controller level. The controller can functionally compare the sensor data to target and historical levels by configuring such levels as threshold settings in the controlled 608. The controller may execute one or more system controls 610 in response to the sensor data and may communicate the sensor data to the application server via communications interface 616. The application server processes the IAQ data 612 and queries and updates the database 614. In an alternative embodiment, the system collects sensor data 604 via the IAQ monitoring devices and processes the sensor data 606 at the App Server 612 level. An application residing in the server can functionally compare the sensor data to target and historical levels by configuring such levels as threshold settings in the controlled 608. The application may execute one or more system controls 610 in response to the sensor data and may communicate the sensor data to the application server via communications interface 616. The application server processes the IAQ data 612 and queries and updates the database 614.

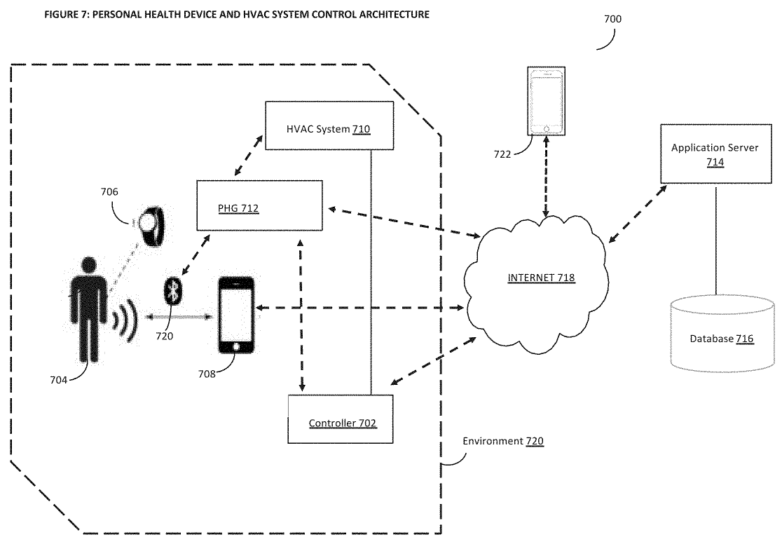

Referring now to FIG. 7, a system architecture of a luminosity, temperature, IAQ, and an environmental control system 700 is shown. According to an embodiment of the present disclosure, the environmental control system 700 generally comprises a smart HVAC controller 702, one or more occupant 704 having a one or more Personal Health Device (PHD) 706, one or more mobile phone 708, an HVAC system 710 operably engaged with controller 702, a wireless Personal Health Gateway (PHG) router 712, and an application server 714 operably engaged with an application database 716. Controller 702 is installed in the interior of a commercial or residential building and may be configured to control one or more functions of HVAC system 710 and to control one or more specific environment 720 occupied by one or more occupant 704. In various embodiments, controller 702 may be configured as a Wi-Fi enabled thermostat, including one or more integrated luminosity, temperature and IAQ sensors. Controller 702 may communicate with application server 714 via one or more communication means (e.g., wireless, cellular, Ethernet, etc.), shown as bi-directional dotted arrows; to and over Internet connection 718. In various embodiments, controller 702 may communicate with PGH 712 via one or more communication means (e.g., Bluetooth, BLE, Wi-Fi, LAN, etc.). In various embodiments, one or more PHDs may communicate with one or more PHG 712 via Bluetooth 720. In various embodiments, one or more PHGs 712 may communicate with HVAC system 710 via wireless including Bluetooth, BLE, Wi-Fi, or the like. In alternative implementations, one or more PHDs 712 may communicate with one or more mobile phone 708 to enable data transmission and reception to application server 714 and subsequently data storage within database 716. In alternative implementations, mobile phone 708 serves as a personal health/wellness/activities device, collecting data of occupant 704 for transmission and reception to application server 714 and subsequently data storage within database 716.

An object of the present disclosure is the Device Management (DM) of PHD 706 and PHG 712 for interoperability (syntactic and semantic) with one or more controllers, HVAC system, and application server. Interoperability is essential for communicating occupant health/wellness/activities information between PHDs and backend applications. In various embodiments, DM of PHDs and PHGs employs one or more Personal Connected Health Alliance (PCHA)-Continua standards or architecture (includes ISO/IEEE 11073, Bluetooth SIG and BLE) to ensure end-to-end, plug-and-play interoperability of PHDs for seamless, secure collection, transmission, and storage of occupant health/wellness/activities data. In various embodiments, DM of PHDs and PHGs employs OMA LWM2M standards to ensure end-to-end, plug-and-play interoperability of PHDs for seamless, secure collection, transmission, and storage of occupant health/wellness/activities data. In various embodiments, each PHD or PHG possesses a unique and uniform resource ID (e.g., UUID format) for registration into the application server. In various embodiments, application server 714 monitors the status of one or more PHD 706 and PHD 712. In a preferred embodiment, an application residing in PHG 712 comprises a DM, a LWM2M client, and Interworking proxy/converter. The DM provides interfaces to enable communication with one or more PHD 706. The LWM2M Client provides interfaces to enable communication with application server 714, preferably a LWM2M server. The Interworking proxy/converter facilitates the conversion of IEEE 11073 protocol attributes into LWM2M objects, vice versa, for interoperability. In various embodiments, PHG 712 monitors and retrieves data (e.g. object instance) from one or more PHDs (e.g. BLE, IEEE 11073 device) through one or more corresponding interfaces. In various embodiments, PHD 706 acts as an Agent (per IEEE 11073 protocol) and PHG 712 acts as a DM. In various embodiments, IEEE 11073 one or more attributes of PHD 706 are mapped into one or more LWM2M device management objects.

Each PHD 706 or mobile phone 708 of one or more occupant 704 may be operably engaged with each other to communicate data to the controller via one or more network topology, including but not limited to mesh, ad-hoc mesh, star, or the like. In various embodiments, a PHD comprises one or more sensors/detector/system, fitness tracker, motion detector, accelerometer, pressure, MEMS sensor, photodetector, ultrasound transducers, microphone, infrared sensor, photodiode, magnetometer, GPS sensor, for sensing, detecting, or monitoring, invasively, minimally invasive, or non-invasively, one or more environmental markers, biomarkers, medical diagnostic markers, biological markers, physiologic markers, electrophysiologic signals, including but not limited to luminosity, temperature, humidity, gas, organic volatile compounds, IAQ, UV, infrared radiation, body fluid constituents, blood constituents, respiration gas (e.g., O2, CO2, ketones, etc.), blood glucose, ISG glucose, urine glucose, electrolytes, constituents of one or more chem panel (e.g. Chem12, etc.), ECG, EKG, EEG, constituents of sweat, lactate, heart rate, steps, motion, speed, acceleration, fall detection, oximetry, pulse oximetry, blood gas (e.g., O2, CO2, etc.), blood pressure, or the like. In various embodiments, the PHD may collect data from an interventional or treatment device (e.g. asthma inhaler, insulin pump, medication dispenser, etc.); whereby time, frequency, or duration of device usage may signal an occupant's health/wellness/activities status. In various embodiments, the location of one or more occupant 704 within a commercial building or a residential structure may be identified or geo-fence using one or more PHD 706, one or more mobile phone 708, or one or more PHG 712. One or more PHD 704 are configured to continuously collect data and communicate such data to application server 714. Application server 714 is preferably a HIPAA compliant server. One or more applications on server 714 processes occupant related data via application logic, and routes PHD data to database 716.

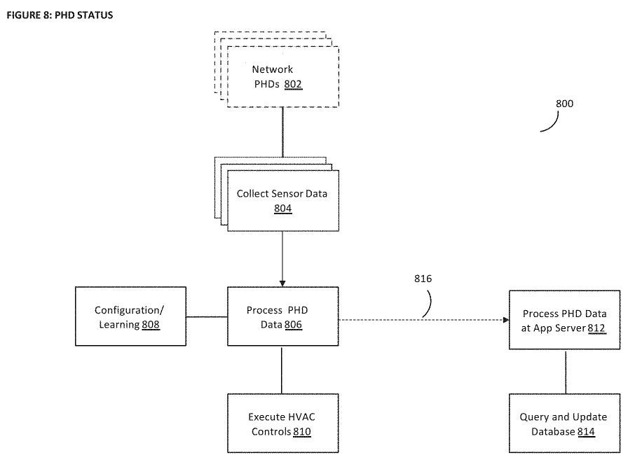

Database 716 aggregates and stores PHD data and associates it with a date/time stamp and a device, occupant, or combinations thereof. Application server 714 is operable to query database 716 to assemble one or more occupant health/wellness/activities reports comprising status, analysis, and diagnostics. The one or more health/wellness/activities reports are assembled in a user interface and accessed via a web browser or mobile interface on a client device 722. In an alternative embodiment, occupant 704 may access health/wellness/activities reports using mobile phone 708. Embodiments of system 100a are operable to execute routines 800 through 1000, as described in FIGS. 8-10 below.

Referring now to FIG. 8, a functional block diagram of a routine 800 for an occupant's health/wellness/activities status is shown. According to an embodiment of the present disclosure, routine 800 for occupant health/wellness/activities status is initiated to collect and communicate from one or more PHD to determine one or more environmental conditions or occupant comfort within a commercial or residential structure. One or more occupants having one or more PHD 802 are located within one or more rooms of the commercial or residential building. In various embodiments, one or more PHDs may operate independently or may be configured or paired to engage with one or more independent PHD via a mesh network, and sensor data is collected and communicated to a PHG controller 804. In one embodiment, the sensor data is processed at the PHG controller level 806 and is communicated to the application server via communications interface 816. The application server processes the PHD data 812 and queries and updates the database 814 with the data. The controller may also execute system configurations and/or machine learning steps 808 in response to the sensor data. System configurations and/or machine learning steps may be utilized to execute one or more HVAC controls 810 via the PHG controller. For example, if an occupant's temperature is elevated or the usage of an asthma inhaler has increased for various reasons, at certain times of day, the controller may execute instructions for the HVAC system to run the fan or increase the fresh air intake to proactively maintain desired comfort or health/wellness/activities condition level at pre-determined thresholds in response to data trends.

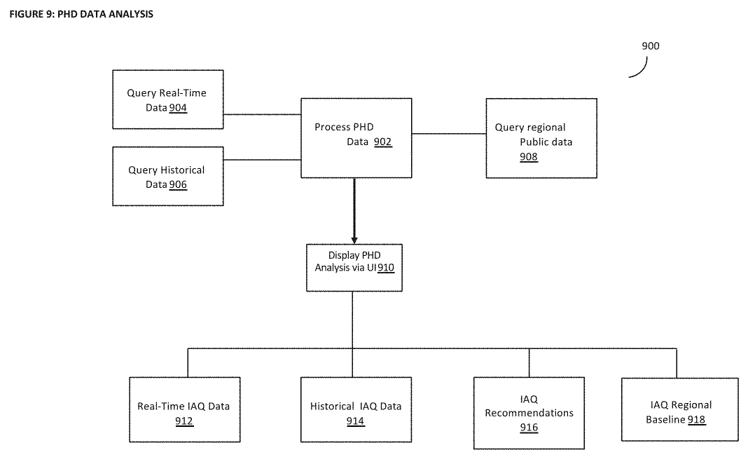

Referring now to FIG. 9, a functional block diagram of a routine 900 for PHD data analysis is shown. According to an embodiment of the present disclosure, routine 900 for PHD data analysis is initiated to process PHD data at the application server level 902. The application server executes instructions to query PHD data from the application database 904, equivalent to database 812, and query historical PHD data from the application database 906, equivalent to database 814. The application server may also query regional or geographically proximal third-party public health data 908 to assess any potential public health trends or scenarios. The application server processes the queried PHD data 902 to assemble an environmental condition analysis for a specific residential or commercial building and communicates and displays the analysis via a user interface 910 executing on a client device. The environmental analysis provides one or more recommendations and data visualizations for the user, including real-time IAQ data 912, historical IAQ data for the subject building 914, and IAQ data recommendations based on real-time and historical data 916, and IAQ comparisons between the subject building and regional baseline data 918.

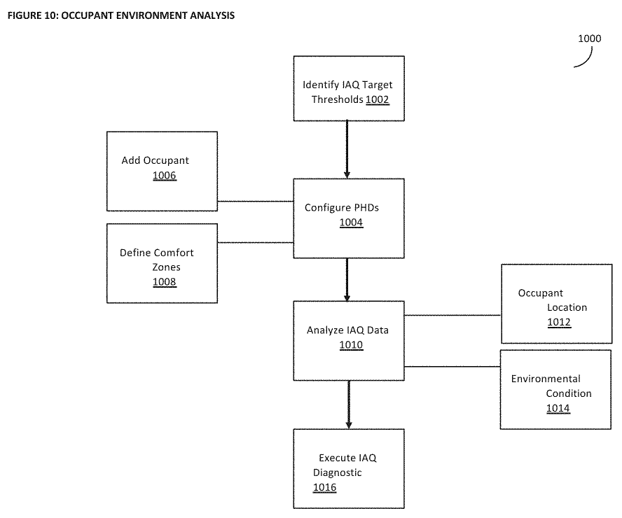

Referring now to FIG. 10, a functional block diagram of a routine 1000 for PHD data analysis is shown. According to an embodiment of the present disclosure, routine 1000 is initiated to execute a PHD data analysis in response to the PHD data analysis of routine 900, as discussed above. Routine 1000 comprises identifying target thresholds for one or more luminosity, temperature, and IAQ measurements (e.g. PM2.5 (particulate matter), carbon dioxide, volatile organic compounds, smoke, and the like) to control a comfort level or environmental condition for one or more occupant. One or more PHDs are configured by one or more occupants residing in one or more rooms of the residential or commercial building 1004 to measure health/wellness/activities data from one or more occupants. If recommended pursuant to routine 900 above, additional occupants and their PHDs can be added 1006, and additional comfort zones can be defined 1008 and associated with the PHDs to provide location-specific environmental quality monitoring. The system analyzes the IAQ data 1010 and aggregates the data according to occupant location 1012 and environmental condition 1014. The IAQ data is processed and an IAQ diagnostic 1016 is then executed by the application server in communication with the application database. In various embodiments, controller 702 queries database 716 for data generated from diagnostic 1016 and automatically adjusts HVAC system 710 to achieve a desired environmental condition set by an occupant. In various embodiments, one or more said HVAC controller, gateway controller, HVAC system, application server, database, or combinations thereof, execute automatic environmental control using one or more said real-time system and methods of the present disclosure.

The above detailed description includes references to the accompanying drawings, which form a part of the detailed description. The drawings show, by way of illustration, specific embodiments in which the invention may be practiced. These embodiments are also referred to herein as “examples.” Such examples may include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein may be machine or computer-implemented at least in part. Some examples may include a computer-readable medium or machine-readable medium encoded with instructions operable to configure an electronic device to perform methods as described in the above examples. An implementation of such methods may include code, such as microcode, assembly language code, a higher-level language code, or the like. Such code may include computer readable instructions for performing various methods. The code may form portions of computer program products. Further, in an example, the code may be tangibly stored on one or more volatile, non-transitory, or non-volatile tangible computer-readable media, such as during execution or at other times. Examples of these tangible computer-readable media may include, but are not limited to, hard disks, removable magnetic disks, removable optical disks, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments may be used, such as by one of ordinary skill in the art upon reviewing the above description.

The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment.

Thus, the following claims are hereby incorporated into the Detailed Description, with each claim standing on its own as a separate embodiment, and it is contemplated that such embodiments may be combined with each other in various combinations or permutations. The scope of the invention should be determined with reference to the appended claims, along with the full scope of equivalents to which such claims are entitled.

What is claimed is:

1. An indoor air quality control system comprising:
a controller being operably engaged with an HVAC system, the controller having a wireless communications interface;
a plurality of indoor air quality sensors operably engaged with the controller via a mesh network, the plurality of indoor air quality sensors being operable to continuously measure one or more indoor air quality data inputs and communicate the one or more indoor air quality data inputs to the controller;
an application server operably engaged with an application database, the application server being communicably connected to the controller via the Internet connection, the controller being operable to communicate the one or more indoor air quality data inputs to the application server in real-time via the Internet connection, and the application server being operable to process the one or more indoor air quality data inputs and communicate the one or more indoor air quality data inputs to the database according to one or more application logic instructions; and,
a client device communicably engaged with the application server, the client device being operable to run an instance of an indoor air quality application via a web or mobile interface, the indoor air quality application being configured to deliver indoor air quality status, analysis, and diagnostics to the web or mobile interface via the indoor air quality application according to the one or more indoor air quality data inputs.

2. The system of claim 1 wherein the one or more indoor air quality data inputs are selected from the group consisting of luminosity, temperature, humidity, particulate matter, carbon dioxide, ozone, volatile organic compounds, sound, light intensity, and smoke.

3. The system of claim 2 wherein the controller is configured to execute one or more HVAC system controls in response to the one or more indoor air quality data inputs from the plurality of indoor air quality sensors.

4. The system of claim 1 wherein the application server is configured to assemble one or more HVAC system control prompts in response to one or more indoor air quality data trends, and communicate the one or more HVAC system control prompts to the controller.

5. The system of claim 4 wherein the application server is configured to execute one or more API to aggregate a plurality of third-party indoor air quality data.

6. The system of claim 1 wherein the controller further comprises a smart phone.

7. The system of claim 5 wherein the application server is further configured to process the one or more indoor air quality data trends and the plurality of third-party indoor air quality data to define one or more indoor air quality remediation outputs.

8. An indoor air quality control system comprising:
a controller being operably engaged with an HVAC system, the controller having a wireless communications interface;
a plurality of indoor air quality sensors operably engaged with the controller via a mesh network, the plurality of indoor air quality sensors being operable to continuously measure one or more indoor air quality data inputs and communicate the one or more indoor air quality data inputs to the controller;
a transceiver hub communicably engaged with the controller and the plurality of indoor air quality sensors;
a system control interface operably engaged with the transceiver hub and the controller;
an application server operably engaged with an application database, the application server being communicably engaged with the transceiver hub via a wireless communications interface, the transceiver hub being configured to communicate the one or more indoor air quality data inputs to the application server in real-time via the wireless communications interface, and the application server being configured to process the one or more indoor air quality data inputs and communicate the one or more indoor air quality data inputs to the database according to one or more application logic instructions; and,
a computing device communicably engaged with the transceiver hub and the application server, the client device being operable to run an instance of an indoor air quality application via a web or mobile interface, the indoor air quality application being configured to deliver indoor air quality status, analysis, and diagnostics to the web or mobile interface via the indoor air quality application according to the one or more indoor air quality data inputs.

9. The system of claim 8 wherein the plurality of indoor air quality sensors are selected from the group consisting of luminosity sensors, temperature sensors, humidity sensors, particulate matter sensors, carbon dioxide sensors, ozone sensors, volatile organic compound sensors, sound sensors, light intensity sensors, and smoke sensors.

10. The system of claim 8 wherein the transceiver hub is configured to control one or more communications protocols of the mesh network.

11. The system of claim 10 wherein the controller is configured to execute one or more HVAC system controls in response to the one or more indoor air quality data inputs from the plurality of indoor air quality sensors.

12. The system of claim 10 wherein the transceiver hub is configured to assign a date/time stamp and device identifier to the one or more indoor air quality data inputs.

13. The system of claim 12 wherein the application server is configured to define one or more HVAC system controls in response to a plurality of indoor air quality historical data.

14. An occupant-centric environmental control system comprising:
a controller being operably engaged with an HVAC system, the controller having a wireless communications interface;
a plurality of indoor air quality sensors operably engaged with the controller, the plurality of indoor air quality sensors being operable to continuously measure one or more indoor air quality data inputs and communicate the one or more indoor air quality data inputs to the controller;
a personal health device being configured to measure personal health or wellness data from a user;
a personal health gateway device operably engaged with the personal health device, the personal health device being configured to communicate the personal health or wellness data to the personal health gateway device, the personal health gateway device being configured to communicate the personal health or wellness data to the controller; and,
an application server operably engaged with an application database, the application server being communicably engaged with the personal health gateway device and the controller via a wireless communications interface to receive the personal health or wellness data and the indoor air quality data and store the personal health or wellness data and the indoor air quality data in the application database, the application server being configured to process the health and wellness data and the indoor air quality data to define one or more environmental correlations between the health and wellness data and the indoor air quality data, and application server being configured to define one or more HVAC system controls according to the one or more environmental correlations.

15. The system of claim 14 wherein the personal health device is a body-worn device.

16. The system of claim 14 the application server is configured to assign a date/time stamp and device identifier to the health and wellness data and the indoor air quality data, and store the date/time stamp and device identifier in the application database.

17. The system of claim 14 wherein the controller is operable to execute one or more HVAC system controls in response to the health and wellness data and the indoor air quality data.

18. The system of claim 14 further comprising a smart phone communicably engaged with the personal health device and the application server, the smart phone being configured to display one or more data visualizations associated with the personal health or wellness data and the indoor air quality data.

19. The system of claim 14 wherein the personal health device is an interventional or treatment device.

20. The system of claim 14 wherein the application server is a HIPPA compliant server.
